(19) United States
(12) Patent Application Publication
(10) Pub. No.: US 2023/0280242 A1
(43) Pub. Date: Sep. 7, 2023

(54) DISAGGREGATION DEVICE

(71) Applicant: Achilles Therapeutics UK Limited, London (GB)

(72) Inventors: Toby Proctor, London (GB); Riccardo Rayan Siena, London (GB); Joanne Wei-Gnan Tang, London (GB); Duncan Young, Melbourn (GB); Simon Waddelow, Melbourn (GB); Michelle Griffin, Melbourn (GB); Samuel Woodhouse, Melbourn (GB); John Briant, Melbourn (GB); Matthew Parker, Melbourn (GB)

(21) Appl. No.: 18/019,152

(22) PCT Filed: Aug. 4, 2021
(86) PCT No.: PCT/GB2021/052010

§ 371 Date: Feb. 1, 2023

(30) Foreign Application Priority Data
Aug. 4, 2020 (GB) 2012117.4

(51) Int. Cl. G01N 1/28; B02C 18/18

(52) U.S. Cl. CPC G01N 1/286; B02C 18/186; G01N 2001/2873

(57) ABSTRACT

The present invention relates to an apparatus for the disaggregation of tissue. The apparatus comprises a first cutting zone comprising a first cutting blade configured to rotate about a first rotational axis, and an opening for the ingress of tissue. The opening is oriented at an angle to the first rotational axis.

### FIELD AND BACKGROUND

The present invention relates particularly, but not exclusively, to an apparatus for the disaggregation of tissue. Specifically, an invention described herein may be used in applications where it is desirable to store, transport and chop human tissue into a size appropriate for later processing and use. The tissue may be from a tumour.

In order for tissue to be processed or tested, it must first be disaggregated to form pieces each sized so as to be suitable for such processing/testing. It has been discovered that a size of 1-3 mm is most appropriate for later processing. In some applications, tissue pieces with a size of 1 to 6 mm or 0.5 to 10 mm may be suitable. If the tissue pieces are too small, then they are less optimal for later processing.

Having been removed from the body of a patient, the pieces of tumour or other tissue may be of varying shapes and sizes. A system is therefore required which is able to handle various shapes and sizes of tumour and reduce them to the desired shape and size for later processing.

Tumour infiltrating lymphocyte (TIL) expansion is a common use of processed tumour pieces which requires pieces of tumour with a consistent size. There is therefore a desire to be able to provide an apparatus which is able to reduce tumour chunks of varying sizes to such a size that they can be used as the starting material for TIL expansion. This is done by disaggregating or chopping the tumour.

Existing systems for disaggregating tumours require complicated operating steps by health care professionals, do not provide a closed system for storage and transportation of the tumour and/or do not result in consistent size pieces of tissue. When in depth or complicated operating steps are required by a health care professional, mistakes can often be made which leads to the tissue not being usable for later processing and therefore being wasted. Furthermore, using systems which are not closed increases the risk of tissue contamination, again leading to wasted tissue. Existing surgical morcellators often axially pull a tissue and draw it along a rotational axis for morcellation. This results in tissue pieces with varying sizes and shapes thus meaning it is not usable for TIL expansion.

The inventors have identified an alternative way in which to disaggregate the tumour in order to achieve uniform shape and size pieces, thus providing the optimum starting material for TIL expansion. The novel apparatus developed by the inventors can be easily and simply operated by a single operator/clinician or the like. The apparatus provides a closed system for the storage and processing of tumour pieces. Therefore, the clinician is able to add the tumour into the apparatus in which it is transported to a laboratory for disaggregation and later processing. This novel system mitigates the risk of tumour pieces removed from a patient being contaminated so that they cannot be used for further processing and investigation. The operation of the device is simple, reducing the possibility of error and thus wasted tumour samples.

It will be recognised from the disclosure herein that the invention is also suitable for disaggregation of other tissues. For example, the invention is suitable for any application that requires disaggregation but not homogenisation of tissues. One such application is the generation of tissue fragments that may subsequently be used as scaffolds for 3-dimensional models for drug screening.

### SUMMARY

Particular aspects and embodiments are set out in the appended claims.

Viewed from a first aspect, there is provided an apparatus for the disaggregation of tissue. The apparatus comprises a first cutting zone comprising a first cutting blade configured to rotate about a first rotational axis, and an opening for the ingress of tissue, the opening being oriented at an angle to the first rotational axis.

The apparatus for the disaggregation of tissue may also be referred to as the tissue disaggregation portion. In effect the apparatus functions as a device for disaggregating or morcellating tissue for use in tumour infiltrating lymphocyte (TIL) expansion. The orientation of the opening at an angle to the first rotational axis increases the efficiency of this disaggregation and enables greater control to be exerted over the rate at which the tissue is presented to the cutting edge of the cutting blade. The angle may be between 15 to 90 degrees from the axis of rotation.

The first cutting blade may be helical. The use of a helical blade is beneficial as the helical flutes aid in clearing disaggregated tissue away from the opening for the ingress of tissue. This prevents clogging of the cutting blade. High helix angles provide a better cutting action on soft materials.

The opening may be oriented substantially perpendicularly to the first rotational axis. Thus the tissue is fed onto the helical blade perpendicular to the direction of motion of the helical blade. Again, this enables greater control to be exerted over the rate at which the tissue is presented to the cutting edge of the cutting blade.

The apparatus may additionally comprise a second cutting zone comprising a second cutting blade. The second cutting blade may be configured to rotate about a second rotational axis. The use of two cutting zones means that the tissue can be disaggregated into pieces smaller than can be achieved with a single cutting zone as the tissue is passed through the first cutting zone which chops the tissue into smaller pieces before passing through the second cutting zone which cuts it into smaller pieces still.

The second cutting blade may be helical. As with the first cutting zone, the helical blade is beneficial as the helical flutes aid in clearing disaggregated tissue away from the blade. The helical first and second cutting blades may both be right handed, both be left handed or may be oriented in opposite directions. Having the helical blades oriented in different directions aids movement of the cut samples through the cutting zones.

The first and second rotational axes may be coaxial. This reduces the space taken up by the cutting zones and allows for both cutters to be driven by a single motor shaft without complex gearing and coupling. The first and second cutting blades or portions may be formed from a single piece of material or may be formed of separate pieces.

The first and second cutting blades may be configured in use to be rotated about their respective axes by one or more drive mechanisms. The use of drive mechanisms enables the cutting blades to be rotated at high speed. This enables more effective disaggregation of the tissue.

The one or more drive mechanisms may be configured to rotate the first and second cutting blades in the same or in opposite directions.

The first cutting blade may have a larger diameter than the second cutting blade. This makes the first cutting blade more suitable for receiving the initial larger piece or pieces of tissue to disaggregate it into smaller pieces which may then be passed through the smaller diameter cutting blade. The use of the smaller diameter cutting blade means the smaller pieces can be disaggregated into even smaller pieces. Thus the tissue can be disaggregated to pieces with shape and size appropriate for further processing.

The helical blade of the first cutting blade may have a first pitch and the helical blade of the second cutting blade may have a second pitch, wherein the first pitch may be greater than, smaller than or equal to the second pitch. When the first cutting blade has a larger pitch than that of the second cutting blade, the depth of cut of the tissue can be increased compared to that with the second cutting blade.

The apparatus may additionally comprise a housing, wherein the first cutting zone and the second cutting zone may be disposed within the housing.

The first cutting zone and the second cutting zones may comprise fluid inlets. These fluid inlets may be used to flush fluid through the disaggregation portion. This prevents clogging of the blades by the tissue as well as preventing the cutting blades overheating due to friction. If a single cutting zone is used, a single fluid inlet may be used.

The fluid inlets may be adjacent to the one or more cutting blades. Thus the fluid can be flushed directly onto the blades. If a single cutting zone is used, the fluid inlet is adjacent the single cutting zone.

The tissue may be sequentially passed through the first cutting zone followed by the second cutting zone. The first cutting zone can therefore disaggregate the tissue into a number of pieces which are then disaggregated further into smaller pieces in the second cutting zone.

The first cutting zone may comprise a first inlet and a first outlet for the tissue and the second cutting zone may comprise a second inlet and a second outlet for the tissue, wherein a conduit may connect the first outlet to the second inlet. Thus the tissue may travel from the first cutting zone through the conduit to the second cutting zone.

The second inlet may be configured in use to communicate tissue into the second cutting zone at an angle to the second rotational axis of the second cutting blade.

The second inlet may be configured in use to communicate tissue into the second cutting zone substantially perpendicular to the rotational axis of the second cutting blade.

The housing additionally may comprise an opening adjacent to the first cutting zone for a thermo electrical connection or an infrared conductive/transparent material. This enables the user to monitor the temperature of the cutting blade whilst the disaggregation is happening. If an infrared conductive/transparent material is used, an infrared thermometer can be used to enable temperature monitoring of the cutting zone.

Viewed from a further aspect, there is provided a tissue processing device comprising a lid and a base. The base is configured to be attached to the lid. The base comprises an outer casing, a tissue receiving portion disposed within the outer casing, and the apparatus for the disaggregation of tissue disposed within the outer casing.

The device may be configured to function as an integrated collection, storage and disaggregation device for tissue. The device may therefore provide a closed system thus preventing contaminants contacting tissue during transportation or disaggregation.

The lid may be configured to be removably attached to the base. Thus, a health care professional can remove the lid to add the tissue before reconnecting the lid to the base.

The tissue processing device may comprise a ratchet located on one of the tissue receiving portions and the lid, wherein the ratchet is configured to prevent the removal of the lid from the base. Thus, when the health care professional reattaches the lid to the base, it is possible for the lid to be attached, for example by screwing, so that the ratchet engages and the lid can no longer be removed. This prevents contamination of the tissue or leakage of any fluid inside the device.

A portion of the outer casing may be configured to interact with a portion of the tissue receiving portion to removably attach the two together. The outer casing therefore protects the internal tissue receiving portion and tissue disaggregation portion and provides an easy to handle device for a health care professional. The outer casing can then be easily removed when the device reaches the laboratory for connecting to a processing system.

The lid may comprise a protruding portion configured to extend into the tissue receiving portion when the device is assembled and the protruding portion may comprise a hydrophobic filter. This may comprise a pore size of 2 μm thus providing a sterile barrier between the tissue receive portion and the interior of the lid. The protruding portion forces the added tissue downwards into the tissue receiving portion towards the disaggregation portion. The hydrophobic filter allows the passage of air to prevent pressure building up when the lid is being attached to the outer casing and when the unit as a whole is subjected to external pressure variation such as during air shipment, whilst preventing the leakage of transport medium from the device into the lid.

Viewed from a still further aspect, there is provided a method of use of the tissue processing device. The method comprises adding tissue to the tissue processing device, docking the tissue processing device in a processing system, operating the apparatus for the disaggregation of tissue, and extracting disaggregated tissue from the tissue processing device.

The method may additionally comprise, before operation of the apparatus for the disaggregation of tissue, removing transport medium from the device, and priming the device with processing fluid. This is to enable the system to operate as a fixed volume system.

The tissue added into the tissue processing device may be configured to be disaggregated by the apparatus for the disaggregation of tissue into pieces that are of about the same size. This is advantageous for the subsequent processing of tumour infiltrating lymphocytes.

The tissue added into the tissue processing device may be configured to be disaggregated by the apparatus for the disaggregation of tissue into pieces having a size of between about 0.5 to about 10 mm. This ensures that the pieces generated are small enough to pass through the downstream fluidic system, but not so small that any cellular content is completely washed from the tissue or subjected to excessive shear stress which would make the cellular content unusable as the starting material for TIL expansion. This size of tissue pieces can be achieved with one or two cutting zones.

The tissue added into the tissue processing device may be configured to be disaggregated by the apparatus for the disaggregation of tissue into pieces having a size of between 1.5 and 5 mm. This is the optimum range of sizes to achieve the above benefits.

Other aspects will also become apparent upon review of the present disclosure, in particular upon review of the Brief Description of the Drawings, Detailed Description and Claims sections.

### BRIEF DESCRIPTION OF THE DRAWINGS

Teachings of the disclosure will now be described, by way of example only, with reference to the accompanying drawings in which:

FIG. 1a shows the tissue processing device of the invention;

FIG. 1b shows a cross sectional view of the tissue processing device of the invention;

FIG. 1c shows a part of the tissue receiving portion of the invention;

FIG. 2a shows the tissue processing device of the invention with the lid removed;

FIG. 2b shows a cross sectional view of the tissue processing device of the invention with the lid removed;

FIG. 3a shows the tissue processing device of the invention with the outer casing removed;

FIG. 3b shows a cross sectional view of the tissue processing device of the invention with the outer casing removed;

FIG. 4a shows the tissue processing device of the invention with the lid and outer casing removed;

FIG. 4b shows a cross sectional view of the tissue processing device of the invention with the lid and outer casing removed;

FIG. 5a shows a top view of the tissue disaggregation portion of the invention;

FIG. 5b shows a cross-sectional side view of the tissue disaggregation portion of the invention;

FIG. 6a shows a side view of the tissue disaggregation portion of the invention;

FIG. 6b shows a cross-sectional top view of the tissue disaggregation portion of the invention;

FIG. 7 is a graph showing the relationship between tumour piece size (particle minor axis length) and the flow rate of processing fluid into the tissue disaggregation portion; and

FIG. 8 is a graph showing the relationship between tumour piece size (particle minor axis length) and the diameter of a first cutting blade (cutter) of the tissue disaggregation portion.

While the disclosure is susceptible to various modifications and alternative forms, specific example approaches are shown by way of example in the drawings and are herein described in detail. It should be understood however that the drawings and detailed description attached hereto are not intended to limit the disclosure to the particular form disclosed but rather the disclosure is to cover all modifications, equivalents and alternatives falling within the spirit and scope of the claimed invention.

As used in this specification, the words “comprises”, “comprising”, and similar words, are not to be interpreted in an exclusive or exhaustive sense. In other words, they are intended to mean “including, but not limited to”.

It will be recognised that the features of the above-described teachings of the disclosure can conveniently and interchangeably be used in any suitable combination. It will also be recognised that the invention covers not only individual embodiments but also combinations of the embodiments that have been discussed herein.

### DETAILED DESCRIPTION

The present teaching relates to a tissue processing device for storage, transportation and disaggregation of tissue. The device may also be used for the disaggregation of other substances.

An embodiment will be described in which the design of the device allows the storage and disaggregation of tissue for use in, for example, isolation and expansion of tumour infiltrating lymphocytes (TIL).

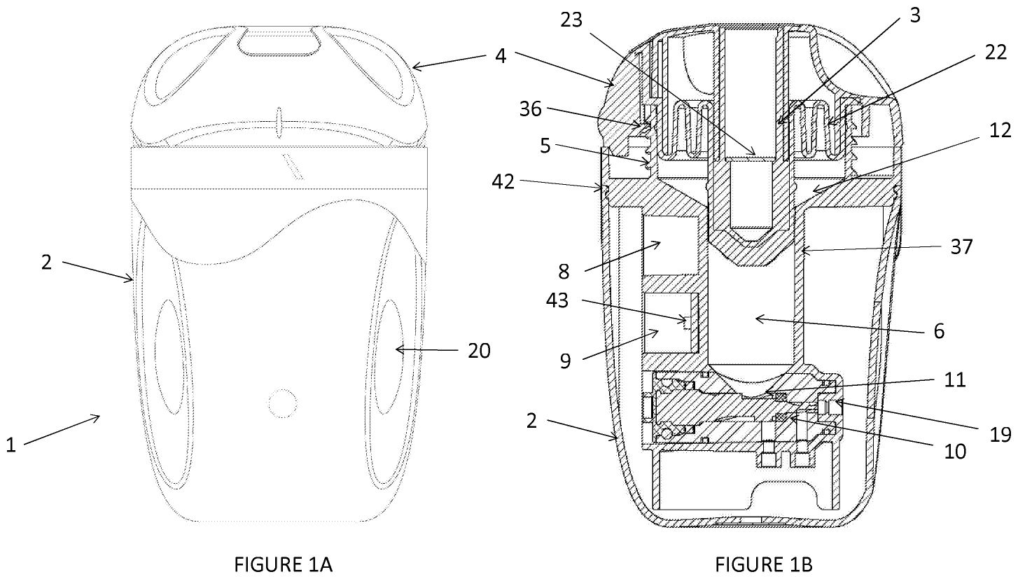

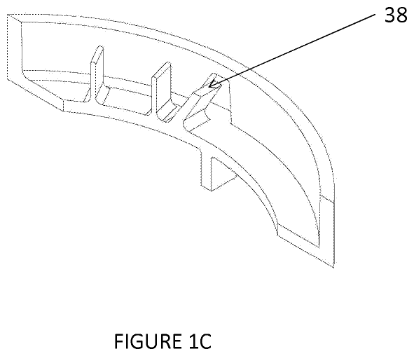

FIG. 1 shows a device 1 according to the invention. FIG. 1A shows a side view of the tissue processing device and FIG. 1B shows a cross sectional view of the tissue processing device. The device comprises a lid 4 attached to a base, wherein the base comprises an outer casing 2. The outer casing 2 has a viewing window 20 through which internal components of the device may be seen.

From FIG. 1B, the internal components of the device can be seen. The lid 4 comprises a lip 36 attachable to a screw fit 5 on the base of the unit.

In the present teaching, the lid 4 comprises a plunger or protruding portion 3 which protrudes into the base of the device when the lid 4 is attached to the base. In the present teaching, the protruding portion 3 has a tapered end.

A hydrophobic filter 23 is positioned across the diameter of the protruding portion 3 of the lid 4. In this way, air is able to travel through the filter but liquids are prevented from traveling through the opening and up the protruding portion 3 of the lid 4. The lid 4 also has an opening to provide a single leak path for air (not shown). This is formed at the top of the top of the lid. A leak path is also formed in the side of the protruding portion (not shown). In this way, when the device is assembled as shown in FIG. 1B, air can travel from an inner collection space 6 (discussed further later), through the leak path in the side of the protruding portion. In the present teaching, the leak path is slit shaped. Thus a build-up in pressure is prevented when the lid is attached to the base.

The protruding portion acts as a plunger. When the tissue has been inserted and the lid attached to the base, a separate tool may be used to deploy the protruding portion further into the inner collection space 6 to force the tissue downwards. The inner collection space 6 can be viewed through the viewing window 20 so that the location of the tissue inside the inner collection space 6 can be monitored. The tapered end of the protruding portion is shaped to be received by an opening 11 in a tissue disaggregation portion 10, discussed further later. When the protruding portion is deployed in the plunging motion, the leak path in the side of the protruding portion is in contact with the inner surface of the inner collection space 6. Thus, fluid is prevented from leaking through this path. This causes an increase in pressure in the inner collection space 6 which helps force the tissue into the cutting portion.

The lid 4 may additionally comprise a rolling diaphragm seal 22 located between the outer radial portion of the protruding portion 3 and the outer radial edges of the lid, wherein one end of the rolling diaphragm seal is attached to the protruding portion of the lid. When the protruding portion is deployed as a plunger in order to force the tissue into the opening 11, the rolling diaphragm seal 22 enables a seal to be maintained as the end of the seal attached to the protruding portion moves downwards with the protruding portion.

The device additionally comprises a tissue receiving portion 37. In the present teaching, the outer cover 2 is attachable to the tissue receiving portion 37 by a protruding lip 42 which interacts with part of the outer cover 2. When an inwardly directed pressure is applied to the outer cover 2 about halfway along its length, the upper edge is forced outwards and released from the lip 42. In this way, the outer cover 2 is removable from the lid 4 and tissue receiving portion 37.

The tissue receiving portion 37 of the present teaching comprises a funnel shaped portion 12 for receiving tissue samples or other materials requiring disaggregation. In some teachings, this portion has markings on its inner surface (not shown). The funnel shaped portion 12 is connected to a top of the inner collection space 6 into which the protruding portion 3 extends when the device is assembled.

The inner collection space 6 contains a transport medium (not shown). The markings on the inner surface of the funnel shaped portion allow a health care professional to monitor the volume of tissue which has been added to the device by monitoring the change in level of the transport medium.

The bottom of the inner collection space 6 is connected to the disaggregation portion 10. Tissue or other substances requiring disaggregation is configured to be fed into the disaggregation portion 10 through the opening 11. In the present teaching, the opening 11 is funnel shaped. This enables the centring of the pieces of tissue before they enter the disaggregation portion 10. Once the tissue has been disaggregated, it leaves the disaggregation portion through outlet 19. The disaggregation portion 10 will be discussed further later.

The tissue receiving portion 37 has at least one docking feature. In the present teaching, the tissue receiving portion 37 has two docking features 8, 9. The two docking features are used to ensure the device is docked at the correct orientation and aligned correctly. One of the docking features may include a magnetic feature 43. These are located on the outer radial side of the inner collection space. These will be discussed further later.

FIG. 1C shows a portion of the tissue receiving portion. The portion has a ratchet feature 38 for interacting with a part of the lid 4 (not shown). Once the lid 4 is screwed onto the tissue receiving portion 37 using the screw fit 5 and the ratchet 38 has engaged with the part of the lid, the lid 4 cannot be unscrewed from the tissue receiving portion 37 due to the interaction of the lid with the ratchet portion.

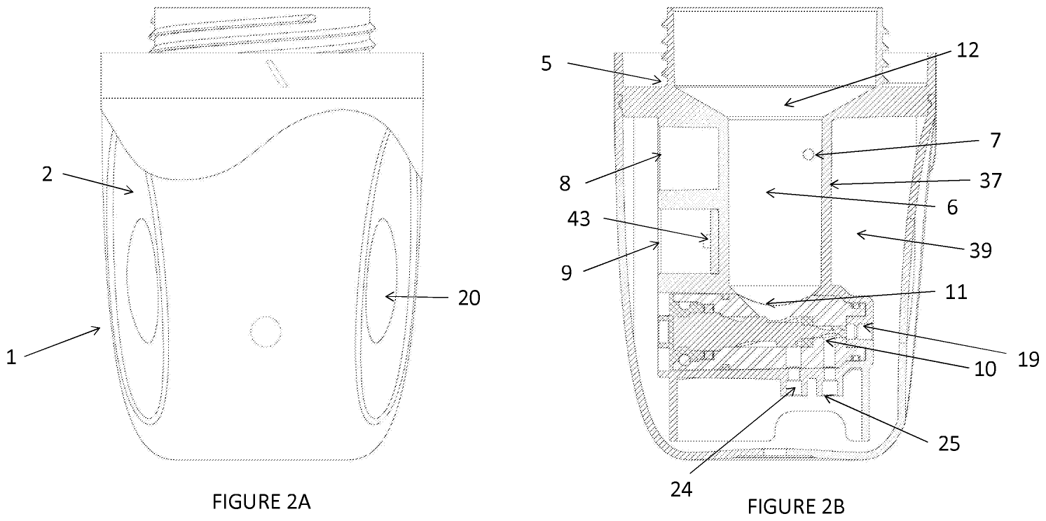

FIGS. 2A and 2B show a side view and cross sectional view respectively of the unit 1 without the lid 4. From the cross sectional view of the device 1 in FIG. 2B, an inlet 7 can be seen through which processing medium flows into the inner collection space 6 when disaggregation of the tissue is being carried out. Tubing (not shown) is attached to the inlet 7 for the passage of transport medium. The tubing is stored in the tubing storage space 39. The disaggregation portion 10 additionally comprises a primary and secondary flushing conduits 24, 25 for flushing processing medium through the disaggregation portion 10 when in use.

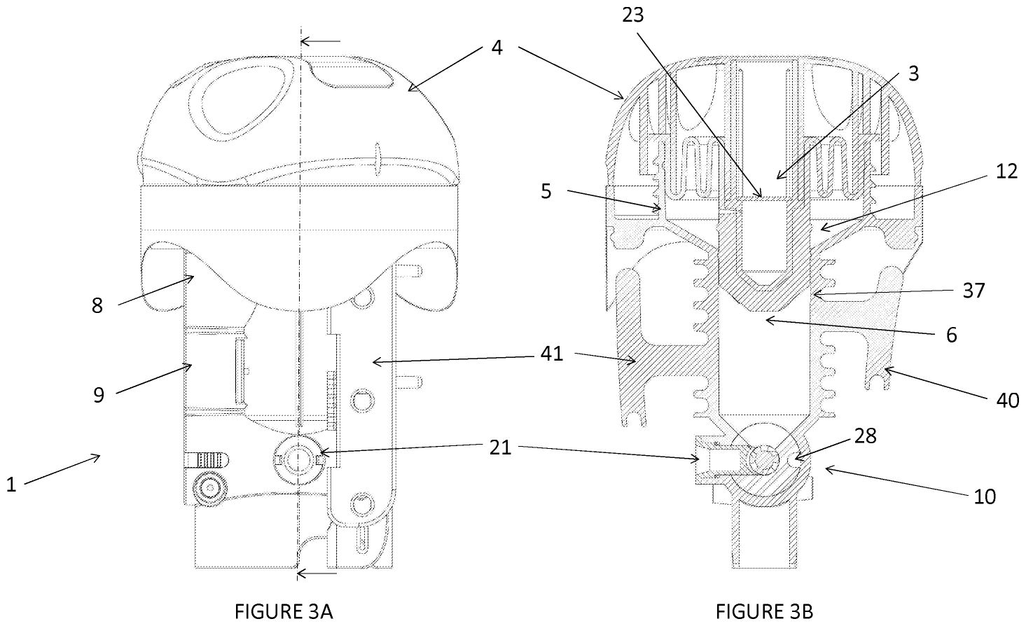

FIGS. 3A shows a side view of the unit 1 with the outer casing 2 removed and with the lid 4 attached. FIG. 3B shows a cross sectional view of the device 1, with the outer casing 2 removed and with the lid 4 attached, in the direction of the arrows shown in FIG. 3A.

As can be seen from these figures, the tissue receiving portion 37 may comprise tubing holding portions 40, 41 attached to its outer radial surface. When the device is in the form as shown in FIG. 1, the tubing attached to the opening 7 is wound around the tubing holding portions 40, 41. The disaggregation portion 10 may have an infrared temperature monitoring window 21, discussed further later.

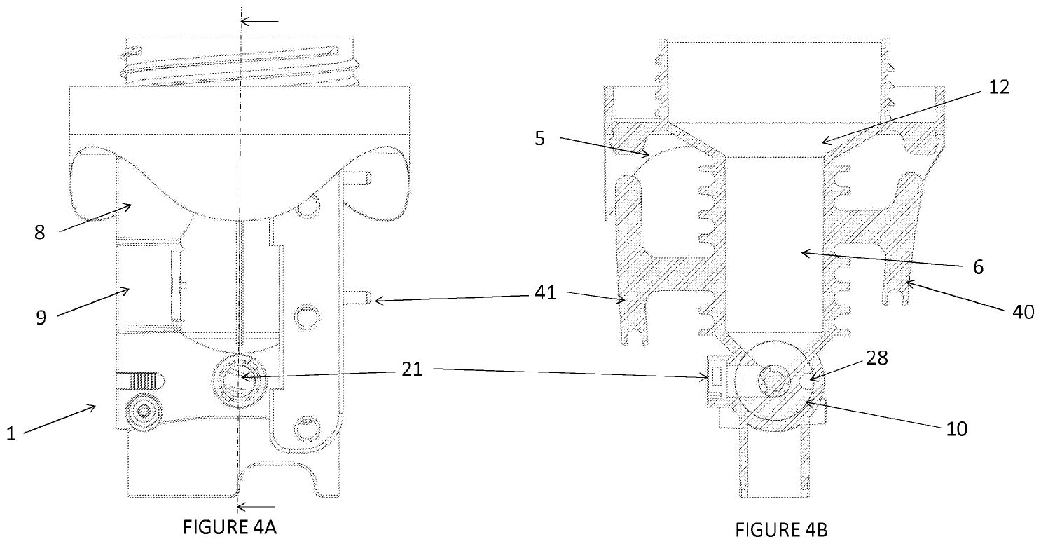

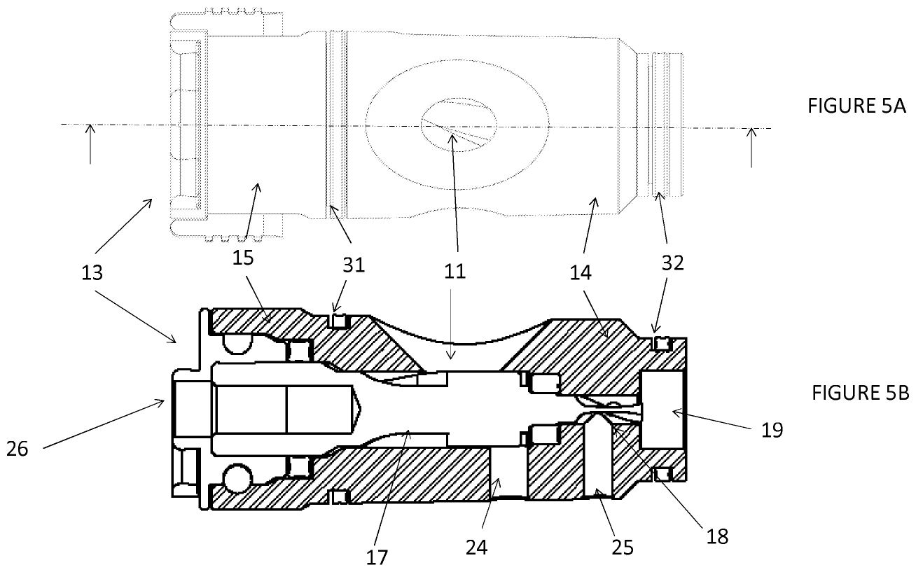

FIGS. 4A and 4B show the views of FIGS. 3A and 3B with the lid 4 removed.

FIGS. 5A and 5B show the disaggregation portion 10. FIG. 5A shows a top view of the disaggregation portion 10. FIG. 5B shows a cross sectional view of the disaggregation portion 10 in the direction of the arrows shown in FIG. 5A.

The disaggregation portion 10 comprises at least one cutting portion. In the present teaching, the disaggregation portion 10 comprises two cutting portions; a first cutting portion 17 and a second cutting portion 18. In some teachings, the disaggregation portion 10 has a single cutting portion.

In the present teaching, the tissue is configured to be passed through the first cutting portion 17 followed by the second cutting portion 18. As can be seen from the figure, the first cutting portion 17 has a larger diameter than the second cutting portion 18.

The disaggregation portion 10 of the present teaching comprises a housing 13. A first portion 14 of the housing encases the at least one cutting portion. A second portion 15 of the housing encases a drive gear 26, discussed further later. A seal 31, for example an O ring seal, is positioned between the first and second housing portions 14, 15. This prevents leakage of transport medium or processing medium from the disaggregation portion 10.

The opening 11 through which the tissue enters the disaggregation portion is formed in the first housing portion 14. In the present teaching, the opening 11 has a circular or oval shape when viewed from above, see FIG. 5A. Tissue is fed into the first cutting portion 17 through the opening 11.

In the present teaching, the first and second cutting portions 17, 18 are rotatable about coaxial rotational axes. In the present teaching, tissue is fed into the first cutting portion 17 in a direction substantially perpendicular to the axes of rotation. In other teachings, tissue may be fed into the first cutting portion at a different angle to the axes of rotation. For example, the tissue may be fed into the first cutting portion at an angle of between 15 and 90 degrees to the axes of rotation.

In the present teaching, each of the first and second cutting portions 17, 18 comprise helical blades. In the present teaching, the pitch of the first helical blade is larger than the pitch of the second helical blade. For example, the pitch of the first blade may be 52 mm and the pitch of the second blade may be 22 mm. In other teachings, the pitch of the first helical blade may be from 26 mm to infinite wherein when the pitch is infinite, the blade is parallel to the rotational axis of the cutting portion. The pitch of the second helical blade may be from 11 mm to infinite. In other teachings, the pitch of the first blade may be from 30 mm to 70 mm or more preferably from 40 mm to 60 mm and the pitch of the second blade may be from 5 mm to 40 mm or more preferably from 10 mm to 30 mm. The blade pitch is chosen so as to prevent clogging of the tissue in the blade. The diameter of the cutting blade may be between 0.5 and 16 mm. In some examples, the diameter may be between 4 and 8 mm.

The helices of the helical blades of the present teaching have opposite ‘handedness’. Specifically, one of the two helical blades is right handed and the other of the two helical blades is left handed. This enables the tissue to be driven from the inlet to the outlet of each cutting portion. In the present teaching, the first helical blade is a right handed blade and the second helical blade is a left handed blade and the helical blades are rotated anticlockwise by the drive gear 26.

In the present teaching, each of the helical blades has two flutes. In other teachings, each helical blade may have more than two flutes. For example, each blade may have up to five flutes.

In other teachings, other non-helical blades may be used. For example, as discussed above, a rotating shaft with one or more blades running along its length (i.e. with infinite pitch) may be used. Alternatively, a blade with right angle cut outs may be used.

The first and second cutting portions 17, 18 are configured to be rotated about their respective axis using the drive gear 26. The cutting portions may be configured to rotate at a speed of between 1 and 1000 rpm. In some examples, the cutting portions may be configured to rotate at a speed of between 50 and 200 rpm.

Primary and secondary flushing conduits 24, 25 are connected to each of the first and second cutting portions 17, 18 respectively. Processing medium enters the disaggregation portion through the opening 11 and passes over each of the blades. At each cutting stage, further processing medium is flushed through tubing connected to conduits 24, 25. This prevents clogging of the cutting blades and prevents overheating due to friction. The processing medium may be pumped into the disaggregation portion through the conduits 24 and 25 at a speed of 0.1-2 litres/minute. In some examples, the speed may be 0.2 to 1 litres/minute. The fluid flow rate is limited to minimise foaming of the fluid caused by protein content in the fluid.

The first housing portion 14 is attached to a part of the tissue receiving device 37. A second seal 32, for example an O ring seal, is formed between the first housing portion 14 and the part of the tissue receiving device 37.

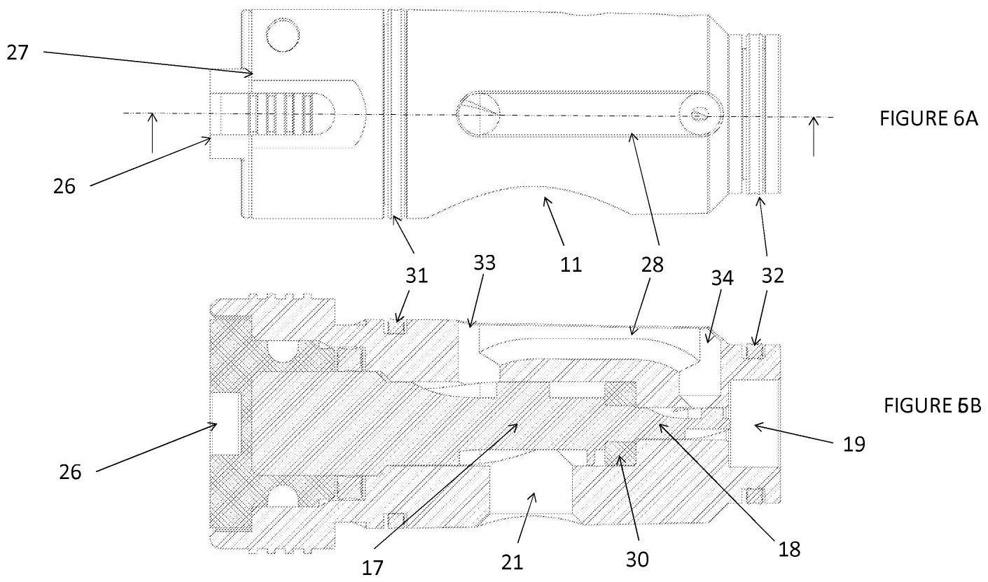

FIGS. 6A and 6B also show the disaggregation portion 10 removed from the device 1. FIG. 6A shows a side view of the disaggregation portion 10. In this figure, the disaggregation portion is shown with the opening 11, which is located at the top of the disaggregation portion 10 when it is installed in the device 1, facing downwards. FIG. 6B shows a cross sectional view of the disaggregation portion viewed in the direction marked on FIG. 6A.

As can be seen from these figures, a conduit 28 extends from a first cutting zone outlet 33 to a second cutting zone inlet 34.

The conduit 28 is formed from a groove in the first housing portion 14. The groove is installed within part of the tissue receiving portion 37 such that an inner surface of the tissue receiving portion combines with the groove to from the conduit 28. This is shown best in FIGS. 3B and 4B. The anticlockwise rotation of the right handed first helical blade causes the tissue entering the opening 11 to be moved to the left whilst being disaggregated by the first blade in order to exit through first cutting zone outlet 33. The anticlockwise rotation of the left handed second helical blade then causes the tissue entering the inlet 34 to be moved to the right whilst being disaggregated in order to exit through outlet 19. In this way, the ‘handedness’, i.e. whether the blades are right handed or left handed, controls the movement of the tissue through the tissue disaggregation portion.

As discussed above, in other teachings, the first helical blade may be left handed and the second helical blade may be right handed. In this case, an alternative location of the tissue inlets and outlets is used to that shown in the figures due to the different direction of movement of the tissue once it enters the disaggregation portion.

Once tissue is inserted into the disaggregation portion 10 through the inlet 11, it passes through the first cutting portion 17. The helical cutting blade cuts the tissue into smaller pieces. The tissue then leaves the first cutting portion through the first cutting portion outlet 33 and travels along the conduit 28 to the second cutting portion inlet 34. At this stage, the tissue pieces are directed into the second cutting portion 18. In the present teaching, the pieces are directed onto the blade substantially perpendicular to the angle of rotation. As with the first cutting portion 17, in other teachings, tissue may be fed into the second cutting portion at a different angle to the axes of rotation. For example, the tissue may be fed into the second cutting portion at an angle of between 15 and 90 degrees to the axes of rotation.

The tissue pieces are cut into smaller pieces by the second cutting blade. The disaggregated pieces then leave the second cutting portion through the tissue outlet 19. Tubing (not shown) is connected to the outlet 19 to carry the disaggregated tissue away.

When a disaggregation portion 10 with a single cutting portion is used, the tissue enters the first cutting portion 17 as is shown in FIGS. 5A and 5B through opening 11. Rather than being passed to a second cutting portion, the tissue pieces leave the first cutting portion through tissue outlet 19. A single conduit 24 is used to flush processing medium onto the first cutting portion 17. The processing medium then travels through the outlet 19 with the disaggregated tissue.

The disaggregation portion 10 may have an infrared temperature monitoring window 21. This is shown in FIG. 6B. In this way, when the cutting blades are running, a user can monitor the temperature of the blades. In the present teaching, the temperature monitoring window is made of High Density Polyethylene (HDPE). HDPE is transparent to Infra-Red radiation in the 10 μm range required to monitor for black body emission around 20-40° C. Thus, the use of HDPE means that a user can ‘see’ the surface of the transfer medium and the first cutting blade since they emit Infra-Red photons as the temperature increases. In other teachings, alternative thermo electrical connections for temperature read out may be used in order to monitor the temperature of the one or more cutting blades or the encompassing housing 13.

A description of a method of using the device will now be explained.

A health care professional receives the device in its form shown in FIG. 1A. The lid 4 is screwed onto the outer casing 2 to hold it in place without the ratchet feature 38 engaging with the lid. The health care professional takes off the lid 4 and places the tissue in the funnel shaped portion 12 so that it sinks into the tissue receiving portion 6. As discussed above, the inner surface of the funnel shaped portion may have markings to enable the health care professional to record the volume of tissue placed in the device. The health care professional then screws the lid 4 onto the base of the device. In the present teaching, and as shown in FIG. 1C, the ratchet feature 38 is preceded by two protruding parts. Thus, the health care professional can be sure that the lid 4 is securely connected to the outer casing 2 upon hearing or feeling the third click when screwing the lid 4 onto the base 2. As the lid is screwed on, the plunger or protruding portion 3 forces the tissue down into the inner collection space 6.

The device 1 with the tissue inside is then returned to the lab. Once in the lab, the technician removes the outer casing 2 of the device. As discussed above, this is done by the technician squeezing a lower portion of the outer casing 2 so that an upper edge of the outer casing is released from the lip 42. Once the outer casing has been removed, the device is as shown in FIGS. 3A and 3B. The tissue receiving portion 37 is then connected to a processing system using the tubing which is unwound from the tubing holding portions 40, 41 and tubing attached to conduits 24 and 25 and outlet 19. If the technician can see that the tissue has not entered the tissue disaggregation portion 10 through the viewing window 20, the plunger is deployed to force the tissue into the disaggregation portion 10.

The docking features 8, 9 are used to dock the device on a docking port. As has been discussed above, in the present teaching, one of the docking features 9 includes a magnetic feature 43. Once the magnetic feature 43 is brought into contact with a corresponding feature on docking port, it is possible for a motor to be driven which rotates the drive gear 26. This ensures that the motor is only driven once the device is docked correctly.

The tubing connected to inlet 7 and to the conduits 24 and 25 is connected to an integrated four way manifold tube connector (not shown) which is connected to the outlet of a pump and a processing medium vessel. The tubing from outlet 19 is connected to the pump via an elution vessel. This is done by sterile welding.

The elution vessel is also connected to an output vessel. The processing system also includes a bag connected to the elution vessel and the outlet of the pump.

Once the tubing from each of the inlet 7, conduits 24 and 25 and outlet 19 have been connected in the laboratory, the transport fluid within the device 1 is removed into the transport medium vessel by applying a negative pressure to the tubing from inlet 7 and conduits 24 and 25 and clamping the tubing from outlet 19 so that fluid cannot pass through.

The device is then primed with a processing fluid. The processing fluid is pumped from the bag through the elution vessel then into the device. This is added through inlet 7 and conduits 24 and 25 whilst maintaining the clamping on the tubing from outlet 19.

The disaggregation of the tissue is then carried out. The processing fluid causes the tissue to travel into the tissue disaggregation portion. If required, the protruding portion may be deployed into the inner collection space 6 by the technician in order to force the tissue into the tissue disaggregation portion. The motor is driven which rotates the drive gear 26. The one or more cutting blades are caused to rotate by the drive gear 26 which enables the disaggregation of the tissue as has been discussed above.

During the disaggregation, processing fluid is circulated throughout the system. Fluid enters the device 1 via inlet 7 and conduits 24 and 25 and exits the device 1 via outlet 19, to which a negative pressure is applied, along with the disaggregated tissue. The disaggregated tissue enters the elution vessel from which it is transported to the output vessel for further use. The flow rate at which the processing fluid is pumped through the conduits 24 and 25 can be altered to control the size of the pieces of disaggregated tissue. Specifically, if the flow rate is increased, the tissue is chopped into larger pieces.

The invention will now be described, by way of example only, with reference to the following Examples.

### EXAMPLES

### Example 1

### Generation of Tissue Pieces

Swine lymph nodes, anterior cervical, parotid, retropharyngeal, sub-maxillary and trachea-bronchial, were isolated from adult porcine plucks. Nodes were stored in phosphate buffered saline and frozen at −20° C. On the day of investigation, samples were warmed in a water bath at 37° C. until completely thawed. Lymph nodes of approximately 0.5 g in weight were placed inside the inner collection space of the tumour processing device, in phosphate buffered saline. Tissue was then disaggregated in a tissue disaggregation portion with a single cutting portion using a combination of different fluid inlet flow rates of the processing fluid e.g., 0.4 L min⁻¹ or 1.0 L min⁻¹, cutting blade (cutter) diameters e.g., 6 mm or 8 mm or cutting blade (cutter) speeds e.g., 56 rpm, 170 rpm or 876 rpm.

Disaggregated tissue was collected and photographed on an illuminating light plate. Images were analysed using Fiji (ImageJ, Opensource), identifying the minor axis length of the best fitting ellipse encompassing each particle. Minor lengths were then analysed and plotted using GraphPad Prism v9.0.2 (GraphPad Software, La Jolla, USA).

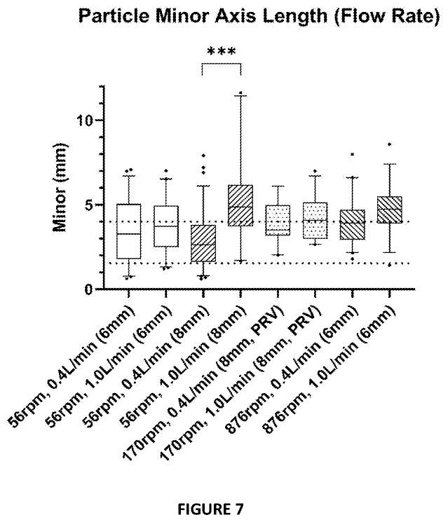

Increasing the fluid flow rate of fluid entering the inlet from 0.4 L min⁻¹ to 1.0 L min⁻¹ resulted in a detectable increase in the median value of the minor axis length of populations of particles generated from swine lymph nodes, where the cutting blade (cutter) speed and diameter were controlled. Increasing the flow rate from 0.4 L min⁻¹ to 1.0 L min⁻¹, with the cutting blade speed set at 56 rpm for an 8 mm cutting blade, resulted in a significant (p <0.001) increase in the median minor axis length from 2.63 mm (n=77) to 4.88 mm (n=20). These results are shown in FIG. 7.

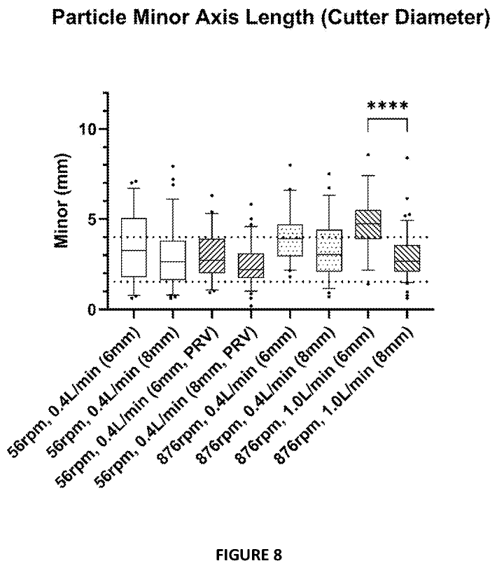

Increasing the cutting blade diameter from 6 mm to 8 mm resulted in a detectable decrease in the median value of the minor axis length of populations of particles generated from swine lymph nodes, where cutting blade speed and fluid flow rate were controlled. Increasing cutting blade diameter from 6 mm to 8 mm, with cutting speed set at 876 rpm and a flow rate of 1.0 L min⁻¹, resulted in a significant (p <0.0001) decrease in the median minor axis length from 4.74 mm (n=30) to 2.68 mm (n=92). These results are shown in FIG. 8.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the spirit and scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. An apparatus for the disaggregation of tissue, the apparatus comprising:
a first cutting zone comprising:
a first cutting blade configured to rotate about a first rotational axis, and
an opening for the ingress of tissue, the opening being oriented at an angle to the first rotational axis.

2. The apparatus of claim 1, wherein the first cutting blade is helical.

3. The apparatus of claim 1, wherein the opening is oriented substantially perpendicularly to the first rotational axis.

4. The apparatus of claim 1, additionally comprising a second cutting zone comprising a second cutting blade, wherein the second cutting blade is configured to rotate about a second rotational axis.

5. (canceled)

6. (canceled)

7. The apparatus of claim 4, wherein the first and second cutting blades are configured in use to be rotated about their respective axes by one or more drive mechanisms.

8. The apparatus of claim 7, wherein the one or more drive mechanisms are configured to rotate the first and second cutting blades in the same or in opposite directions.

9. (canceled)

10. (canceled)

11. (canceled)

12. (canceled)

13. (canceled)

14. (canceled)

15. The apparatus of claim 4, wherein the apparatus is configured in use for the tissue to be sequentially passed through the first cutting zone followed by the second cutting zone.

16. The apparatus of claim 4, wherein the first cutting zone comprises a first inlet and a first outlet for the tissue, wherein the second cutting zone comprises a second inlet and a second outlet for the tissue, and wherein a conduit connects the first outlet to the second inlet.

17. The apparatus of claim 16, wherein the second inlet is configured in use to communicate tissue into the second cutting zone at an angle to the second rotational axis of the second cutting blade.

18. The apparatus of claim 17, wherein the second inlet is configured in use to communicate tissue into the second cutting zone substantially perpendicular to the rotational axis of the second cutting blade.

19. (canceled)

20. A tissue processing device comprising:
a lid; and
a base, wherein the base is configured to be attached to the lid and wherein the base comprises:
an outer casing;
a tissue receiving portion disposed within the outer casing; and
the apparatus of any of claim 1 disposed within the outer casing.

21. The tissue processing device of claim 20, wherein the lid is configured to be removably attached to the base.

22. The tissue processing device of claim 20, wherein a ratchet is located on one of the tissue receiving portion and the lid, wherein the ratchet is configured to prevent the removal of the lid from the base.

23. The tissue processing device of claim 20, wherein a portion of the outer casing is configured to interact with a portion of the tissue receiving portion to removably attach the two together.

24. The tissue processing device of claim 20, wherein the lid comprises a protruding portion configured to extend into the tissue receiving portion when the device is assembled and wherein the protruding portion comprises a hydrophobic filter.

25. A method of use of the tissue processing device of claim 20, the method comprising:
adding tissue to the tissue processing device;
docking the tissue processing device in a processing system;
operating the apparatus for the disaggregation of tissue; and
extracting disaggregated tissue from the tissue processing device.

26. The method of claim 25, additionally comprising, before operation the operating of the apparatus for the disaggregation of tissue:
removing transport medium from the device; and
priming the device with processing fluid.

27. A method of claim 25 wherein the tissue added into the tissue processing device is configured to be disaggregated by the apparatus for the disaggregation of tissue into pieces that are of about the same size.

28. The method of claim 25, wherein the tissue added into the tissue processing device is configured to be disaggregated by the apparatus for the disaggregation of tissue into pieces having a size of between about 0.5 to about 10 mm.

29. The method of claim 25, wherein the tissue added into the tissue processing device is configured to be disaggregated by the apparatus for the disaggregation of tissue into pieces having a size of between 1.5 and 5 mm.
